(19) United States
(12) Patent Application Publication
(10) Pub. No.: US 2008/0027679 A1
(43) Pub. Date: Jan. 31, 2008

(54) Wearable Device, System and Method for Measuring Physiological and/or Environmental Parameters

(76) Inventor: Dror Shklarski, Yavne (IL)

Correspondence Address: EMPK & Shiloh, LLP, 116 JOHN ST,, SUITE 1201, NEW YORK, NY, 10038, US

(21) Appl. No.: 11/658,082

(22) PCT Filed: Jul. 20, 2005
(86) PCT No.: PCT/IL05/00772

§ 371 Date: Jan. 22, 2007

Related U.S. Application Data
(60) Provisional application No. 60/589,581, filed on Jul. 21, 2004.
(60) Provisional application No. 60/589,572, filed on Jul. 21, 2004.

(51) Int. Cl. G06F 11/30; A61B 5/02

(52) U.S. Cl. 702/182; 600/485

(57) ABSTRACT

Systems, devices and methods are provided for monitoring parameters. The system, according to some embodiments, may include a wireless mobile monitoring device with an array of sensors and a safety module to prevent simultaneous sensing selected parameters and charging of the device, and a medical center server enabled to remotely reconfigure the functioning of the monitoring device.

### FIELD OF THE INVENTION

The present invention relates to wearable devices, systems and methods for monitoring and evaluating physiological and/or environmental parameters, and to communication devices, systems and methods for preventing the use of the device while being charged.

### BACKGROUND OF THE INVENTION

Continuously monitoring a patient's physiological condition generally requires the patient's hospitalization, usually at great cost, especially where long term monitoring is required. In certain situations it is possible to monitor the physiology of patients who are physically outside of the hospital, using wearable monitoring devices.

There are, for example, wrist-worn devices that typically record a patient's physiological data, such as the patient's ECG, during a predetermined recording time. These devices may include event recorders that may capture a patient's physiological data during a physiological “event”, such as a cardiac arrhythmia or an episode of patient discomfort. The event recording may be activated manually by the patient or automatically by determining when monitored physiological data meets predefined event criteria.

Wrist-worn devices typically require that a patient return to a medical center periodically or remotely communicate with a medical center in order to transfer the recorded data for interpretation by a medical staff.

### SUMMARY OF THE INVENTION

According to some embodiments of the present invention, a system is provided for monitoring parameters, including a wireless mobile monitoring device with an array of sensors and a safety module to prevent simultaneous sensing of one or more selected parameters and charging of the device, and a medical center server enabled to remotely reconfigure the functioning of the monitoring device.

According to further embodiments of the present invention, a device is provided for monitoring parameters, including an array of sensors, each sensor having a sensor controller, a main controller to enable reconfiguration of the sensor controllers by commands received from a remote server, and a safety module to prevent simultaneous sensing and charging of the device.

According to further embodiments of the present invention, a method is provided for monitoring parameters, including determining if a user of the wearable monitoring device is trying to charge the device while the device is being operated, initiating actions to prevent simultaneous sensing and charging of the device, and when the attempt to charge the device has ceased, returning to normal operation of the device.

According to further embodiments of the present invention, a method is provided for monitoring parameters, including a mechanism to determine if a measurement is in excess of a selected user limit, and when required, to automatically initiate selected additional actions.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of a medical monitoring system according to some exemplary embodiments of the present invention;

FIGS. 2A, 2B, and 2C are schematic illustrations of external top, bottom, and side view layouts, respectively, of a wearable monitoring device according to some exemplary embodiments of the present invention;

FIG. 3 is a schematic illustration of an internal layout of a wearable monitoring device, according to some exemplary embodiments of the present invention; and

FIG. 4 is a flow chart describing a method for preventing sensing of one or more selected parameters with the device, while the device is being charged.

### DETAILED DESCRIPTION OF THE INVENTION

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or to a similar electronic computing device, that manipulates and/or transforms data represented as physical, such as electronic quantities within the computing system's registers and/or memories into other data similarly represented as physical quantities within the computing system's memories, registers or other such information storage, transmission or display devices.

The processes and displays presented herein are not inherently related to any particular apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of embodiments of the invention as described herein.

It should be appreciated that according to some embodiments of the present invention, the method described below may be implemented in machine-executable instructions. These instructions may be used to cause a general-purpose or special-purpose processor that is programmed with the instructions to perform the operations described. Alternatively, the operations may be performed by specific hardware that may contain hardwired logic for performing the operations, or by any combination of programmed computer components and custom hardware components.

Although the scope of the present invention is not limited in this respect, the wearable device disclosed herein may be implemented in any suitable wired or wireless device that may be a handheld, worn, or other suitable portable communications device. By way of example, the wearable devices may include wireless and cellular telephones, smart telephones, personal digital assistants (PDAs), wrist-worn devices, and other suitable wearable devices or any parts of them. Alternatively, according to other embodiments of the present invention, the system and method disclosed herein may be implemented in computers.

Embodiments of the present invention are directed to an improved wearable device, a system, and a method for manually and/or automatically monitoring medical parameters and/or processing data related to medical parameters and/or alerting a patient and/or medical facility regarding a medical condition. For example, the device, system, and method may enable monitoring physiological and/or environmental parameters, sending alerts to a Medical Center (MC) and/or to a patient, processing sensed data, and updating device parameters and/or functions. The wearable device, according to some embodiments of the present invention, may be prevented from being operated while it is being charged, for example, via a charger connected to an electrical network.

The wearable device may independently transfer a patient's physiological and/or environmental data, and/or other suitable data to, for example, the MC, when, for example, certain parameters are above or below predetermined ranges or thresholds that may be defined according to the particular needs of a patient. In some embodiments, the wearable device may also transfer the data to a MC if the parameters are within predetermined thresholds or ranges. In some embodiments, a MC may receive, via a communications channel, a patient's physiological data and additional information, such as, for example, the location of the patient, directly from the wearable device. A person or persons at the MC may remotely update, for example, the ranges or thresholds for determining the status of vital parameters of an individual patient, at the discretion of the staff, for example, medical staff, information technology staff and/or technical/engineering staff in the MC, or a device at the MC may automatically update various parameters. Additionally, the MC may remotely update operational features, for example, changing modes of operation, adding new features, updating the device's software and/or part thereof etc. for a wireless device of an individual patient or for a group of patients. Any wearable or remote device for monitoring, measuring, communicating etc. vital parameter data of a patient to the MC may be used, for example, a wearable device as described in detail below.

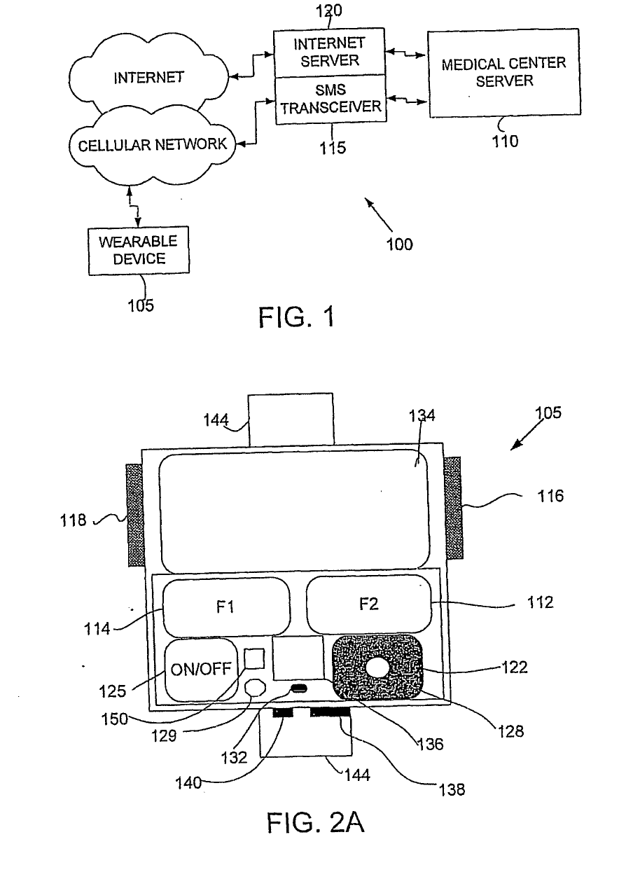

Reference is now made to FIG. 1, which schematically illustrates a medical monitoring system 100 in accordance with some exemplary embodiments of the present invention. Medical monitoring system 100 may include, for example, at least one wearable device 105 that may communicate with a MC server 110. The communication between wearable device 105 and MC server 110 may include wireless data communication, for example, cellular telecommunication (e.g., General Packet Radio Service (GPRS)), satellite communication, wireless LAN, WiFi, Bluetooth, or other suitable communications technologies, and a computer network, for example, the Internet or a local area network (LAN) etc. There may be a plurality of bi-directional and/or uni-directional communication channels between the MC server 110 and wearable device 105, and there may be a plurality of MC servers 110 and/or wearable devices 105 in selected embodiments according to the present invention. Data may be transferred between wearable device 105 and MC server 110 using the above or other suitable means.

In one embodiment the bi-directional communication channel between the MC server 110 and wearable device 105 is a Short Message Service (SMS) channel that may enable communication of data via SMS transceiver 115 to and/or from the wearable device 105, via a cellular communications network. The SMS channel may enable transmission of messages from wearable device 105 to MC server 110, via SMS transceiver 115. In one embodiment the bi-directional communication channel between the MC server 110 and wearable device 105 is an Internet Protocol (IP) based channel, that may enable communication of data via Internet server 120, for example, using File Transfer Protocol (FTP) or other suitable data transfer protocols. In some embodiments a combination of communication networks may be used. For example, if the SMS channel is not available and/or not chosen by the wearable device 105, wearable device 105 may communicate with MC server 110 using FTP. In other embodiments wearable device 105 may communicate with MC server 110 using, for example, SMS and Internet communications. In some embodiments wearable device 105 may communicate with MC server 110 via a Web interface, for example, a Website, where data, commands, and/or requests, etc., may be entered and/or received by wearable device 105 and/or MC server 110.

In one embodiment the bi-directional communication channel between the MC server 110 and wearable device 105 may utilize TCP/IP protocol. In one embodiment a File Transfer Protocol (FTP) may be used to upload physiological data of the patient, e.g., sensed measurement data, from wearable device 105 to MC server 110, and to download data such as updates to software modules from MC server 110 to wearable device 105. Usage of FTP or any other protocol may require the wearable device 105 to logon, for example as an FTP client, to the Internet server 120.

In some embodiments a voice channel, as described below, may be used to enable the staff associated with MC server 110, or suitable software and/or hardware at the MC 100, to communicate with the patient who is using wearable device 105 and/or to enable the patient using wearable device 105 to communicate with the staff at MC server 110.

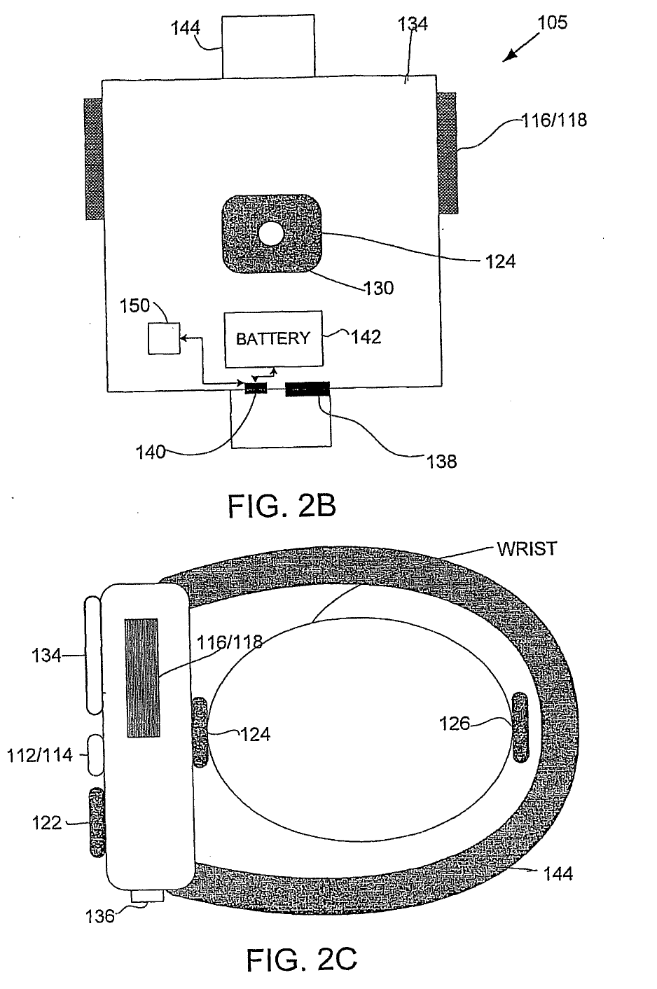

Reference is now made to FIGS. 2A, 2B, and 2C, which schematically illustrate examples of external top, bottom, and side view layouts, respectively, of a wearable device 105 in accordance with some exemplary embodiments of the present invention. Wearable device 105 may include, for example, input components such as functional buttons 112 and 114 for inputting data or commands to operate wearable device 105 (e.g., for approving and/or rejecting of instructions) emergency buttons 116 and 118, that may be used to manually initiate an emergency mode (e.g., by pressing them together or pressing one of them), and an On/Off button 125 to switch wearable device 105 on or off. The On/Off button 125 may be unified with any of the other buttons, for example functional buttons 112 and 114. Wearable device 105 may include one or more electrodes, for example, an ECG RA (Right Arm) finger electrode 122, an ECG LA (Left Arm) wrist electrode 124 (FIG. 2B), and an ECG REF. (Reference) wrist electrode 126 (shown in FIG. 2C). Electrodes 122 and 124 may be located at any suitable location or locations on wearable device 105. For example, electrode 124 may be located on the top side of wearable device 105. In some embodiments, the ECG REF. Wrist electrode 126 may be located at any suitable location on the inner side of wearable device 105 or on the inner side of a strap 144. Wearable device 105 may be worn on a patient's left or right hand or arm, e.g., on the wrist, or on the left or right foot or leg, e.g., on the ankle, and the various components may be appropriately located to enable measuring of parameters on the left and/or right hand and/or arm and/or foot and/or leg.

In some embodiments wearable device 105 may include at least one blood oxygen level (SpO₂) transceiver 128 to measure the level of the oxygen in the patient's blood, and at least one pulse transceiver 130 (shown in FIG. 2B) to measure the patient's pulse. Blood oxygen saturation level (SpO₂) transceiver 128 may be incorporated into, for example, electrode 122 and/or may be independent of electrode 122. In some embodiments wearable device 105 may include a pulse transceiver or sensor 130. The location of pulse transceiver 130 within wearable device 105 may be appropriately positioned to enable sensing of the pulse of the patient. Pulse transceiver 130 may be incorporated into, for example, electrode 124 and/or may be independent of electrode 124. In some embodiments, wearable device 105 may include one or more transceivers, electrodes, or sensors to enable measurement of SpO₂, ECG, pulse, blood pressure data, skin temperature data, respiration data, perspiration data, cardio impedance data, blood sugar or glucose level, and/or other suitable data. The SpO₂, pulse and/or other parameters may be presented on a display area 134 of wearable device 105. The pulse and/or other parameters may be transferred to the MC server 110. Other sensor mechanisms may be used.

Wearable device 105 may include a speaker 136 to enable a patient to hear audio signals, for example from voice communications initiated from MC server 110 or from other sources. When wearable device 105 is operated in a continuous mode of operation, wearable device 105 may, for example, continuously read the pulse of the patient, using pulse transceiver 130. The location of pulse transceiver 130 within wearable device 105 may be appropriately positioned to enable sensing of the pulse of the patient. Pulse transceiver 130 may be incorporated within electrode 124 or may be separate from electrode 124. An indication of the pulse of the patient and/or other parameters may be presented on the display area 134 of wearable device 105. The pulse and/or other parameters may also be transferred to the MC server 110. Other sensor mechanisms may be used. connector 140, the user may not be able to wear wearable device 105 and/or close device strap 144.

In other embodiments device 105 may include a safety module 150, for example, which may be connected to charger connector and/or the main controller of device 105. Safety module 150 may prevent usage of device 105, for example, to sense of one or more selected parameters while device 105 is being charged. For example, the device 105 software and/or hardware may stop the device activities with and/or without notification to the user using for example, display area 134, speaker 136. In other examples the hardware and/or software of the device 105 may cut off the connection of the battery to the electrical circuit and/or cut the operation of the device while the battery is still connected. In another embodiment safety module 150 may prevent charging of device 105 while device 105 is being used. For example, the device hardware and/or software may cut-off the device charger circuits from the charger. In other example the device 105 software and/or hardware may stop the device charging with and/or without notification to the user using, for example, display area 134, speaker 136, of device 105. In another embodiment, safety module 150 may notify the MC server 110 when charging of device 105 while device 105 is being used. The MC server 110 and/or is associated staff may use the communication channels to the device 105 in order to cut off the operation of the device and/or notifying the user using for example, display area 134, speaker 136. The communication channels may be one of the described above.

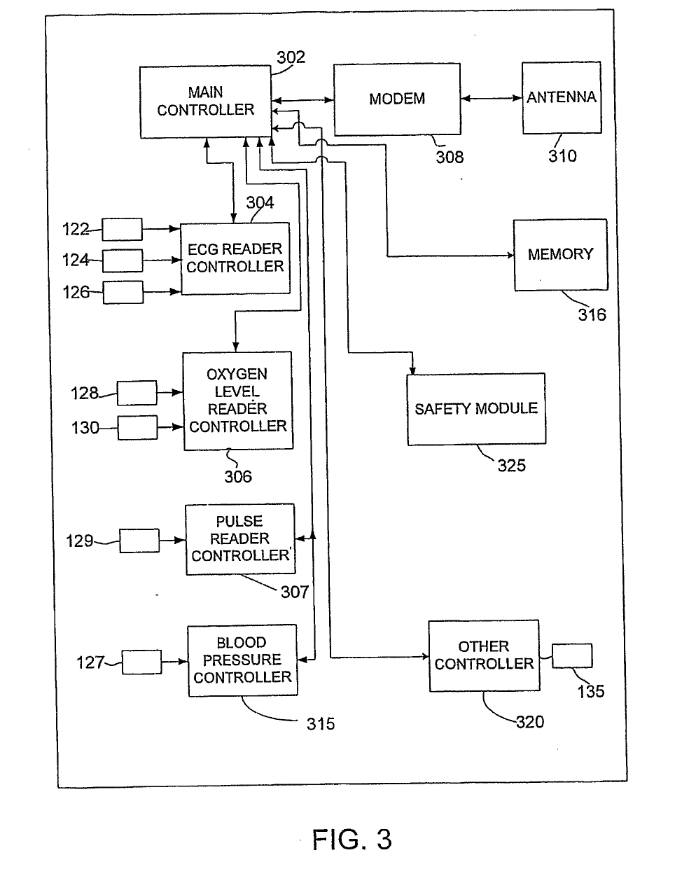

Reference is now made to FIG. 3, which is a schematic illustration of an internal layout of wearable device 105 in accordance with some embodiments of the present invention. Wearable device 105 may include, for example, a main controller 302 to control wearable device operation. Wearable device 105 may include an oxygen level reading controller 306 that may receive input from, for example, one or more SpO₂ transceivers 128 and one or more pulse transceivers 130, or from other sensors or combinations of sensors, and may generate output signals through main controller 302. Wearable device 105 may include a pulse reading controller 307 that may receive input from one or more pulse sensors 129, or from other transceivers or sensors, or

Display area 134 may display additional information such as, for example, medical parameters of the patient, messages received from MC server 110, operational instructions, date and time, parameters that are related to functional elements of wearable device 105 etc. Display area 134 may be, for example, a color display and/or a monochromatic display and may have any desired resolution, depending on the type of data to be displayed. In some embodiments, display area 134 may include an interactive display, for example, a touch sensitive display, and may have a voice activated circuit to control Display area 134. Display area 134 may display any combination of alphanumeric characters, and/or text and/or two-dimensional and/or three-dimensional graphics and/or icons.

Additional elements in wearable device 105 may include one or more service connectors, for example, a service connector 138 that may connect the wearable device 105 to external units such as, for example, a computer, a testing unit, an external medical device, an external display unit, and/or a communication unit, etc. Wearable device 105 may include a charger connector 140 that may be used to connect wearable device 105 to a power source to enable charging of a battery 142 (FIG. 2B). A charger connector 140 may be included in service connector 138. Wearable device 105 may include optional strap 144 that may be used to attach wearable device 105 to the wrist or other location of the patient. Wearable device 105 may include various other suitable components and/or devices, which may be implemented using any suitable combination of elements and components and may incorporate hardware and/or software.

In accordance with some embodiments of the present invention, the charger connector 140 shape and/or placement may prevent the user from connecting device 105 to a charger and/or charger cable, while, for example, the wearable device 105 is being worn by a user. For example, the placement of the charging connector 140 may be on the inner side of strap 144, requiring removal from the user's wrist, foot etc. before charging. Thus, any interaction between wearable device 105 and an electrical network/system while device 105 is being worn may be prevented. In accordance with some embodiments of the present invention, if a charger and/or a charger cable is connected to the charging combinations of transceivers or sensors, and may generate suitable output signals through main controller 302. Wearable device 105 may include a blood pressure controller 315 that may receive input from one or more blood pressure sensors 127, or from other transceivers or sensors, or combinations of transceivers or sensors, and may generate suitable output signals through main controller 302. Wearable device 105 may include, in place of and/or in addition to the above controllers and sensors, additional or alternative controllers 320 (e.g., a blood pressure reading controller, blood sugar level reading controller, temperature reading controller, CI (Cardio Impedance) reading controller etc.), that may receive input from one or more suitable sensor(s) 135 or transceivers, and may generate suitable output signals through main controller 302.

In some embodiments, main controller 302 may receive data from input components, for example, data received from functional buttons 112 and 114, emergency buttons 116 and 118 (e.g., by pressing them together, pressing one of them etc.), On/Off button 125 (e.g., the On/Off button 125 may be unified with any of the other buttons, for example functional buttons 112 and 114), and/or from other components, such as service connector 138, charge connector 140, and battery 142. Main controller 302 may generate outputs that may be transferred to output components, for example, display area 134, speaker 136, a modem, an antenna, or any other suitable output devices.

In some embodiments main controller 302 and/or another unit, sub-system or mechanism may automatically initiate one or more actions in the wireless mobile monitoring device, based on previous measurement(s).

In some embodiments, Oxygen level reading controller 306 may receive signals indicative of vital signs and/or other physiological parameters of the user from sensor 128 and/or 130. Oxygen level reading controller 306 may also receive instruction data, for example via main controller 302, from functionality buttons 112 and 114, emergency buttons 116 and 118, or other suitable sources. Oxygen level reading controller 306 may transfer data, for example via main controller 302, to output components, for example, display area 134, speaker 136, modem, etc.

In some embodiments, Pulse reading controller 307 may receive signals indicative of vital signs and/or other physiological parameters of the user from sensor 129, or other suitable transceivers or sensors. Pulse reading controller 307 may also receive instruction data, for example, via main controller 302, from functionality buttons 112 and 114, emergency buttons 116 and 118, or other suitable sources. Pulse reading controller 307 may transfer data, for example via main controller 302, to output components, for example display area 134, speaker 136, a modem, etc.

In some embodiments, the main controller 302, Oxygen level reading controller 306, pulse reading controller 307, blood pressure controller 315, as well as other controllers if used, for example, a blood sugar level controller and/or other suitable controllers 320 may be implemented in a single controller or in multiple separate controllers or any combinations of controllers.

In some embodiments, wearable device 105 may include sensors and controllers to enable measurement and/or processing of, for example, ECG data, blood pressure data, skin temperature data, body temperature data, respiration data, cardio impedance data, blood sugar or glucose level data, and other suitable data. Respective controllers may receive signals indicative of vital signs and/or other physiological and/or environmental parameters of the user from respective sensors. Respective controllers may receive instruction data, for example via main controller 302, from functionality buttons 112 and 114, emergency buttons 116 and 118, or other suitable sources. Respective controllers may transfer data, for example via main controller 302, to various output components, for example display area 134, speaker 136, a communication modem, etc.

In some embodiments of the present invention, Oxygen level reading controller 306 may receive signals from SpO₂ transceiver 128 and/or pulse transceiver 130, and may receive instruction data signals from main controller 302, from function buttons 112 and 114, emergency buttons 116 and 118, etc. Oxygen level reader controller 306 may generate output signals that may be transferred via main controller 302 to one or more output components of wearable device 105 such as display area 134, and to a communication modem to transfer the data regarding measured parameters to MC server 110 and/or to another destination.

In some embodiments of the present invention, data and signals transferred between the components and modules of wearable device 105 may be transferred in, for example, serial communication lines, I/O lines, and/or other suitable designated lines. For example, a V_{BAT} signal may activate an alert indicating that battery 142 is weak and a V_{CHARGER} signal may activate an alert indicating that battery 142 is charged. Other suitable signals and functions may be implemented.

According to some embodiments of the present invention, device 105 may include a safety module 325, which may be connected to charger connector 140 and/or the main controller 302 of device 105. Safety module 150 may prevent usage of device 105, for example to sense one or more selected parameters, while device 105 is being charged. In another embodiment safety module 150 may prevent charging of device 105 while device 105 is being used. In another embodiment safety module 150 may prevent the charging of device 105 by notifying MC server 110 to remotely prevent the charging of the device 105 while is the device is being used.

In some embodiments, safety module 150 may be implemented as software and/or hardware components in the main controller 302. In some other embodiments of the current innovation, safety module 150 may be implemented fully or partially as mechanical part/s within the wearable device 105. In some other embodiments of the current innovation the memory 316 may be implemented fully or partially as part of the main controller 302. In some other embodiments of the current innovation one or more of main controller 302, ECG reading controller 304, Oxygen level reading controller 306, pulse reading controller 307 and Blood Pressure controller 315, as well as other controllers 320, for example, blood sugar reading controller, temperature reading controller etc. may be implemented in at least a single controller or in multiple separate controllers or combinations of controllers.

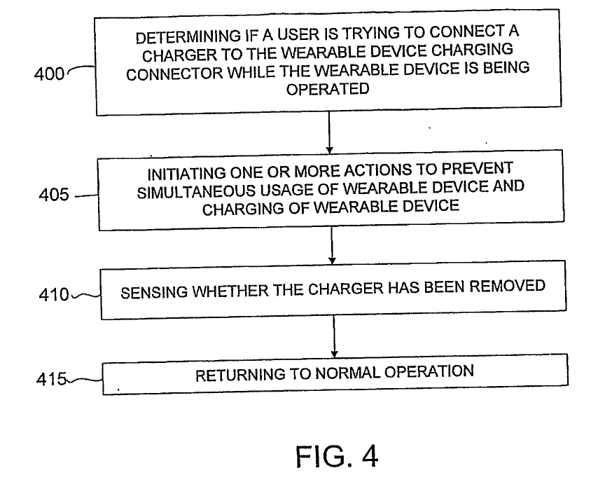

Reference is now made to FIG. 4, which a flow chart describing a method for preventing usage of the device, for example to sense of one or more selected parameters, while it is being charged. For example, wearable device 105 may include safety module 325 to automatically initiate one or more actions to prevent simultaneous sensing and charging of device 105. At block 400 wearable device 105 may determine if a user is trying to charge the wearable device while wearable device 105 is worn and/or is being operated, for example, by connecting a charger to said device. At block 405 the wearable device 105 may initiate one or more actions to prevent simultaneous sensing of device 105 and charging of device 105. For example, safety module 325 (see FIG. 3) may generate a message in display area 134 indicating a visual warning; generate an audible signal using the wearable device speaker 136; reporting and/or alarm the MC server 110 via wireless communication; turn off the charging function, and/or turn off wearable device 105 etc. In one example, the staff of MC server 110 and/or an automated message may instruct the user regarding the danger of charging the device while using it, or may provide alternative instructions or suggestions. The communication between wearable device 105 and MC server 110 may include wireless data communication, for example, cellular telecommunication (e.g., General Packet Radio Service (GPRS)), satellite communication, wireless LAN, WiFi, Bluetooth, or other suitable communications technologies, and a computer network for example, the Internet or a local area network (LAN) etc. In one embodiment the communication channel between the MC server 110 and wearable device 105 is a Short Message Service (SMS) channel that may enable transmission of messages from wearable device 105 to MC server 110, via SMS transceiver 115. In one embodiment the communication channel between the MC server 110 and wearable device 105 is an Internet Protocol (IP) based channel, that may enable communication of data via Internet server 120, for example, using File Transfer Protocol (FTP) or other suitable data transfer protocols. At block 410 device 105 may sense whether the charger has been removed, and/or the user may remove the wearable device 105. At block 415 device 105 may return to normal operation. According to some embodiments of the present invention, the wearable device 105 may act in that ways independently if the charger was connected to the electrical network or not.

According to further embodiments of the present invention, a method is provided for monitoring parameters using mobile wearable device 105, including determining if a measurement is in excess of a selected user limit, and when required, to automatically initiate selected additional actions based on a previous measurement(s).

In accordance with some embodiments of the present invention, medical monitoring system 100 may operate in at least one of a keeper mode, an extended mode, and an emergency mode, or any other appropriate mode, as described below.

The keeper mode may be used as the default mode of wearable device 105, such that wearable device 105 may enter this mode when the device is switched on. Other modes may alternatively be used as the default mode. In the keeper mode, wearable device 105 may, for example, continuously and/or intermittently read the pulse and/or another parameters of a patient. In this mode, wearable device 105 may display parameter data on display area 134, may alert the patient with a message on display area 134, and/or may alert the patient using an audible signal via speaker 136, for example, by playing back predefined audio signals. In addition, wearable device 105 may transmit the measured parameters and/or results from analyses or processing of the measured parameters, to MC server 110, for example, using FTP channel and/or SMS channel. In the event where the staff at MC server 110, or MC server 110 automatically, determines that the patient's pulse is abnormal, according to predetermined criteria or ranges described in detail below, wearable device 105 may alert the patient. According to some embodiments of the present invention, wearable device 105 may determine when one or more parameters are abnormal or, for example, in a danger range. This determination may be instead of or in addition to similar determinations at or by MC server 110. According to some embodiments of the present invention MC server 110 may automatically determine when one or more parameters are abnormal or, for example, in a danger range, instead of or in addition to the staff of MC server 110. Additionally, wearable device 105 may send a warning message to MC server 110, using, for example, the SMS channel, FTP channel etc. When wearable device 105 is operated in the keeper mode, parameters such as, for example, pulse, SpO₂, and ECG may be monitored continuously and/or at selected intervals, for example, every twelve hours, or at other selected intervals.

In the extended mode, wearable device 105 may be set to perform operations according to a pre-defined schedule, for example, to periodically measure oxygen levels in the patient's blood (SpO₂) and/or ECG. In this mode, wearable device 105 may display parameters or other data on display area 134, may alert the patient with a message on display area 134, and/or may alert the patient using an audible signal via speaker 136, for example, by playing back predefined audio signals. In addition, wearable device 105 may transmit the measured parameters and/or results from analyses or processing of the measured parameters, to MC server 110, for example, using a FTP channel and/or a SMS channel. When wearable device 105 is operated in the extended mode, parameters such as pulse, SpO₂, and ECG, may be monitored, for example, five times a day by default. The default may be at shorter or longer intervals. If the staff at MC server 110 or MC server 110 automatically detects, for example, that the heart rate, oxygen level in the blood, and/or ECG records and/or other data are abnormal (e.g., according to pre-defined criteria or ranges as discussed below), wearable device 105 may alert the patient by providing output signals in the display area 134 or via speaker 136. Additionally or alternatively, wearable device 105 may send a message to MC server 110 and/or to another destination, for example, using the FTP channel.

In the emergency mode a patient may initiate operation of the medical monitoring system 100, e.g., by pressing, for example, any of the emergency buttons 116 or 118. When operating in the emergency mode, wearable device 105 may send emergency messages to MC server 110 or to another destination using, for example, the FTP channel. Emergency messages may additionally or alternatively be sent to MC server 110 or to another destination via the SMS channel, for example, in cases where the FTP channel is not available. In addition, when entering an emergency mode, measurement of SpO₂ and/or ECG and/or other parameters may be initiated. The staff of MC server 110 or another source may, in response to entering into emergency mode, initiate a call and/or message to the patient of wearable device 105, or MC server 110 may automatically make a call and/or send a message etc. to the patient of wearable device 105.

Wearable device or mobile medical device 105 may measure one or more physiological and/or environmental parameters. According to the results of the measurements device 105 may activate additional functions. In one embodiment device 105 may measure a user's pulse at pre-defined time intervals and/or continuously. In case the measurement is in excess of pre defined limits of the user (which may be customized for a particular user) at least one of the following activities may be automatically initiated in any order or combination: measure the vital signs that caused the trigger again; measure additional parameters, for example: blood oxygen saturation, 1-lead ECG, blood pressure, temperature etc.; alert the user via the wearable device display; alert the user using the wearable device beeper and or speaker; transmit a message including the measured result and/or possible additional information to MC server 110, by one or more of the following means: a SMS mechanism, which may utilize GSM, GPRS, CDMA or any other cellular method; a cellular network and the Internet network in order to log to MC server 110 and download the message; a cellular network by creating a call between MC server 110 and medical device 105, by MC server 110, using, for example, switched circuit communications or any other wireless communication; and an open voice channel between MC server 110 and device 105 over the cellular network.

In a second embodiment, in addition to the functions described above, wearable device 105 may measure the blood oxygen saturation at pre-defined time intervals and/or continuously, which may trigger the automatic activity described above.

In a third embodiment, in addition to the functions described above, device 105 may measure ECG (e.g., using 1 or more ECG leads) at pre-defined time intervals and/or continuously which may trigger the automatic activity described above.

In a fourth embodiment, in addition to the functions described above, wearable device 105 may measure the blood pressure at pre-defined time intervals and/or continuously which may trigger the automatic activity described above.

In a fifth embodiment, in addition to the functions described above, device 105 may measure any other vital signs at predefined time intervals and/or continuously which may trigger the automatic activity described above.

In a sixth embodiment, in addition to the functions described in the five embodiments described above, device 105 may keep at least one record of the results of the measurements and/or the sent messages in the device memory 316. The memory may be temporarily or may be non-temporary.

In a seventh embodiment, in addition to the functions described in the six embodiments described above, device 105 may also send historical data, for example, one or more historical messages stored in device memory 316, to MC server 110 using one of the communication means.

In an eighth embodiment, the user may push the device emergency button 116 and 118, in which case, according to an embodiment, device 105 may automatically initiate one or more of the following functions in any order and/or combination: Measure vital signs, for example, pulse blood oxygen saturation, ECG, blood pressure, temperature etc.; Confirm the emergency alert to the user via the display; Confirm the emergency alert to the user via the device beeper and/or speaker 136; Transmit a message to MC server 110 by one or more of the following means: a SMS mechanism (e.g., using GSM, GPRS, CDMA or any other cellular method); a cellular network and the Internet network in order to log on to the MC server 110 and download the message; a cellular network by creating a call between the MC and wearable device 105 by the MC server 110 (e.g., using switched circuit communications and/or any other wireless communication; an open a voice channel between MC server 110 and wearable device 105 over the cellular network. Device 105 may keep at least one record of the results of the measurements and/or the sent messages in its memory. The memory may be temporary or non-temporary.

In a ninth embodiment, in addition to the functions described in the eighth embodiment described above, device 105 may also send one or more historical messages that were stored in device memory 316 to MC server 110 using one of the communication means.

In a tenth embodiment, wearable device 105 may measure the user vital signs according its operation mode. For example, according to a pre-defined schedule, a message may be automatically transmitted to MC server 110, including the results of historical measurements and/or suitable additional information, using a SMS mechanism (e.g., it may uses the GSM, GPRS, CDMA or any other cellular method), the cellular network, and the Internet network, in order to log on to MC server 110 and download the message, a cellular network by creating a call between MC server 110 and wearable device 105 by MC server 110 (e.g., using switched circuit communications), and by using any other suitable wireless communication. In another example, one or more of the parameters may be measured again, for example the pulse, blood oxygen saturation, 1-lead ECG, blood pressure, temperature etc. In a further example, the user may be updated or alerted via the display. In an additional example, the user may be updated via the device beeper and/or speaker 136. In some additional examples a voice channel may be opened between the MC server 110 and wearable device 105 over the cellular network. In an additional example, the transmitted measurements may be deleted from device memory 316.

In an eleventh embodiment, in addition to the functions described in the tenth embodiment above, the transmission may be done when the stored old measurements reached and/or passed a selected amount of memory capacity.

In a twelfth embodiment, in addition to the functions described in the eleven embodiments listed above, and if MC server 110 is not able to be contacted by wearable device 105, wearable device 105 may initiate at least one of the following activities in any order and combination: try to transmit the message(s) again later on (e.g., the time interval for trials may be fixed and/or variable), using the same means of communication and/or other means of communication; alert the user via the display; and alert the user via the device beeper and/or speaker 136.

In a thirteenth embodiment, in addition to the functions described in the twelve embodiments listed above, the information being sent to MC server 110 may be managed using Last In First Out (LIFO). Thus, for example, the last measurement recorded may be the first one transmitted to MC server 110. Other modes of data management may be used.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A system for monitoring parameters, comprising:
a wireless mobile monitoring device, said device including an array of sensors, said device including a safety module to prevent simultaneous sensing and charging of said device; and
a medical center server enabled to remotely reconfigure the functioning of said monitoring device.

2. The system of claim 1, wherein the functioning of said array of sensors are remotely configured by said server.

3. The system of claim 1, wherein said array of sensors includes at least one physiological sensor and at least one environmental sensor.

4. The system of claim 1, wherein said array of sensors includes at least two physiological sensors.

5. The system of claim 1, wherein said monitoring device is adapted to perform one or more functions selected from the group consisting of measuring parameters, transmitting parameter data, processing parameter data, analyzing parameter data, initiating device actions, updating parameter settings, providing warnings, providing instructions, providing alerts, transmitting results, saving data, sending historical data, confirming alerts, and managing measurement data using Last In First Out.

6. The system of claim 1, wherein said monitoring device is adapted to function in one or more of keeper mode, extended mode, and emergency mode.

7. The system of claim 1, wherein said monitoring device is adapted to measure one or more selected parameters continuously and/or intermittently.

8. The system of claim 1, wherein said monitoring device is adapted to automatically send a warning message to said medical center server if parameters measured exceed a selected threshold.

9. The system of claim 1, wherein said monitoring device is adapted to automatically initiate one or more actions based on previous measurement(s).

10. The system of claim 1, wherein said remote configuration of said mobile monitoring device includes remotely implementing a customized software update.

11. The system of claim 1, comprising an update module.

12. A mobile device for monitoring physiological parameters, the device comprising:
an array of sensors, each sensor having a sensor controller;
a main controller to enable reconfiguration of said sensor controllers by commands received from a remote server; and
a safety module to prevent simultaneous sensing and charging of said device.

13. The device of claim 12, wherein said array of sensors includes at least one sensor to measure physiological parameters and at least one sensor to measure environmental parameters at pre-defined time intervals and/or continuously.

14. The device of claim 12, wherein said array of sensors includes one or more sensors selected from the group consisting of an ECG sensor, Oxygen level sensor, pulse sensor, sweat sensor, skin temperature sensor, pH level sensor, blood pressure sensor, external temperature sensor, air humidity level sensor, and pollution level sensor.

15. The system of claim 12, wherein said monitoring device is adapted to automatically initiate one or more actions based on previous measurement(s).

16. A method for remotely reconfiguring a monitoring device, the method comprising:
determining if a user of said wearable monitoring device is trying to charge said device while said device is being operated;
initiating actions to prevent simultaneous sensing and charging of said device; and
when said attempt to charge said device has ceased, returning to normal operation of said device.

17. The method of claim 16, comprising transmitting commands to a wireless monitoring device, from a medical center server, to remotely reconfigure settings of said device.

18. The method of claim 16, wherein said wireless monitoring device includes an array of sensors, said sensors enabled to be individually reconfigured by said medical center server.

19. The method of claim 16, wherein said array of sensors includes one or more sensors selected from the group consisting of an ECG sensor, Oxygen level sensor, pulse sensor, sweat sensor, skin temperature sensor, pH level sensor, external temperature sensor, air humidity level sensor, and pollution level sensor.

20. The method of claim 16, comprising remotely initiating one or more actions in said wireless monitoring device, by said medical center server.

21. The method of claim 16, comprising automatically initiating one or more actions in said wireless monitoring device, by the wireless monitoring device, based on previous measurement(s).

22. The method of claim 16, wherein said remote configuration includes implementing customized software updates.

23. The method of claim 16, comprising remotely updating client software in said wireless monitoring device, by said medical center server.

24. The method of claim 16 comprising keeping at least one record of the results of the measurements and/or the sent messages in the device memory.

25. The method of claim 16 comprising managing measurement data using Last In First Out.

26. A system for monitoring parameters, comprising:
a wireless mobile monitoring device, said device including an array of sensors, said device including a safety module to prevent simultaneous sensing and charging of said device; and
a medical center server enabled to communicate with said monitoring device.

27. The system of claim 26, wherein said array of sensors includes at least one physiological sensor.

28. The system of claim 26, wherein said monitoring device is to function in one or more of keeper mode, extended mode, and emergency mode.

29. The system of claim 26, wherein said monitoring device is to measure one or more selected parameters continuously and/or intermittently.

30. The system of claim 26, wherein said monitoring device is to automatically send a warning message to said medical center server if a parameter measured exceeds a selected threshold.

31. A system for monitoring parameters, comprising:
a wireless mobile monitoring device, said device including an array of sensors, said device including a mechanism for automatically initiating one or more actions in said wireless mobile monitoring device, by the wireless mobile monitoring device, based on previous measurement(s); and
a medical center server enabled to communicate with said monitoring device.

32. The system of claim 32, wherein said array of sensors includes at least two physiological sensors.

33. The system of claim 32, wherein said monitoring device is to function in one or more of keeper mode, extended mode, and emergency mode.

34. The system of claim 32, wherein said monitoring device is to measure one or more selected parameters continuously and/or intermittently.

35. The system of claim 32, wherein said monitoring device is to automatically send a warning message to said medical center server if a parameter measured exceeds a selected threshold.
